(19) United States
(12) Patent Application Publication
(10) Pub. No.: US 2025/0090782 A1
(43) Pub. Date: Mar. 20, 2025

(54) Nasal Cannula Assembly

(71) Applicant: Ngah Chai LEOW, PETALING JAYA (MY)

(72) Inventor: Ngah Chai LEOW, PETALING JAYA (MY)

(21) Appl. No.: 18/803,379

(22) Filed: Aug. 13, 2024

(30) Foreign Application Priority Data
Sep. 19, 2023 (MY) UI2023005617

(51) Int. Cl. A61M 16/06; A61M 16/08

(52) U.S. Cl. CPC A61M 16/0672; A61M 16/085; A61M 2230/432

(57) ABSTRACT

A nasal cannula assembly has a manifold body with a septum-like structure that divides into inlet and outlet ports, each defining a lumen having a first length and a first inner diameter. The lumen comprises a protrusion that extends at least partially in the longitudinal direction of the lumen. A channel is disposed proximate to the septum-like structure having a second length, defining an aperture having a second inner diameter, and a pair of nasal prongs. The assembly has a first sealing member with a recess that extends longitudinally such that the recess of the first sealing member conforms to the shape of the lumen, and a second sealing member. The first length of the inlet and outlet ports is equal to the second length of the channel. The second inner diameter of the channel is less than the first inner diameter of the inlet and outlet ports.

### CROSS-REFERENCE TO RELATED APPLICATIONS

This application claims priority to, and the benefit of, Malaysian Utility Model Application No. UI2023005617, filed on Sep. 19, 2023, the contents of which are incorporated by reference herein in their entirety.

### TECHNICAL FIELD

The disclosure relates generally to ventilation devices, and more particularly to a nasal cannula assembly for supplying and sampling gases to a user via the nasal passages.

### BACKGROUND ART

A gas sampling system continuously transports a flow of gas from or to a patient through a tube. A gas sampling system is generally used in hospital settings, for example during certain medical procedures, during anaesthesia or sedation. Medical professionals may wish to provide patients with respiratory assistance in the form of supplemental oxygen or airflow for many reasons in intensive care unit or home environments. Different types of interfaces for supplying gases to patients are available. For instance, various nasal masks, full face masks, oral interfaces, nasal pillows and nasal cannula interfaces are applicable.

Commonly, a nasal cannula is used to provide a flow of gases to a patient via the patient's nostrils or other nasal passages if required. Such device is configured to either deliver oxygen in to or draw carbon dioxide out from the nostrils of the patient. Nasal cannula assemblies generally consist of entry tubing, either symmetric or single sided that lies across the upper lip. Protruding from this tubing are a pair of open-ended prongs which extend into the nostrils of the patient to deliver oxygen. Nasal cannulas are advantageous in the sense that they are more comfortable and acceptable to be used than a face mask to most patients. The technologies related to nasal cannula assemblies are generally established and are disclosed in the prior art.

One example of a nasal cannula configured to deliver a flow of fluid to a patient is disclosed United States of America U.S. Pat. No. 8,161,971B2. Disclosed herein is a patient interface that comprises a body portion configured to communicate with a first fluid path, a first nostril interface extending from the body portion, wherein the nostril interface communicates with the first fluid path, and a securement portion extending from the body portion proximate the nostril interface, wherein the securement portion is constructed and arranged to engage an exterior surface of a patient's nose to secure the patient interface. Another example is disclosed in United States of America U.S. Pat. No. 11,420,002B2 describing a nasal cannula comprising a manifold part having an inlet to receive the flow of fluid and at least one outlet to deliver the flow of fluid to the patient's nostrils, a port located on the manifold part to deliver a medicament into the flow of fluid being delivered by the nasal cannula to the patient. United States of America U.S. Pat. No. 7,353,826B2 also describes a ventilation interface comprising a nasal cannula body having a pair of nasal prongs location on a top portion of the nasal cannula body and a bellows-like structure configured to contact a bottom surface of the nose to create a sealing interface between the nasal cannula body and the nose.

The main drawback of such devices is that the flow of oxygen, carbon dioxide and breathing of the patient may create turbulences adjacent to the opening of the prongs and ports, thereby reducing the overall efficiency of oxygen delivery due to additional dead space thereof. Dead space in a nasal cannula can affect the efficiency of oxygen delivery to the patient. Dead space refers to the volume of air within the nasal cannula system that is not involved in the gas exchange process and does not reach the patient's lungs. When a patient breathes in through a nasal cannula, the inspired air consists of both fresh oxygen and a portion of the exhaled air that remains within the dead space. This re-breathed air, which contains a lower concentration of oxygen, reduces the overall efficiency of oxygen delivery. The higher the dead space volume, the greater the dilution of the inspired oxygen concentration. These presence of unnecessary dead space will affect both the delivery of oxygen and the accuracy of carbon dioxide sampling altogether. As such, there is a need for the prior device to reduce or eliminate the unwanted dead space to prevent the aforementioned drawbacks. The present invention provides such a solution to the drawback.

### BRIEF SUMMARY

One aspect of the invention is to provide a nasal cannula assembly comprising a manifold body having an inlet port and an outlet port with substantially reduced length for minimizing dead space thereof. Advantageously, minimizing dead space of the inlet and outlet ports reduces or eliminates turbulences adjacent to the opening of the inlet and outlet ports, thereby lessens comingling of supplied oxygen and exhaled carbon dioxide to be sampled.

Another aspect of the invention is to provide a nasal cannula assembly comprising the abovementioned inlet port having a protrusion disposed on an inner surface thereof that extends at least partially in the longitudinal direction of its lumen. A sealing member having a recess configured to conform to the shape of the lumen of the inlet port is provided for sealing thereof. Advantageously, the protrusion functions as an indicator for a medical personnel or professional to connect the inlet port to a source of oxygen for delivering oxygen to the nostrils of a patient.

Still, one aspect of the invention is to provide a nasal cannula assembly comprising a channel that extends of the manifold body whereby the channel has an aperture with substantially reduced inner diameter to minimize dead space thereof.

At least one of the preceding objects is met, in whole or in part, in which the embodiment of the present invention describes nasal cannula assembly comprising a manifold body comprising a septum-like structure configured to separate an inlet chamber and an outlet chamber that extend generally axially of the manifold body and internally dividing the manifold body into an inlet port and an outlet port, each defining a lumen having a first length (L1) and a first inner diameter (Ø1), wherein the lumen of the inlet port comprises a protrusion disposed on an inner surface thereof that extends at least partially in the longitudinal direction of the lumen, a channel disposed proximate to the septum-like structure and in fluid connection with the outlet chamber, the channel having a second length (L2) and defining an aperture having a second inner diameter (Ø2) and a pair of nasal prongs having a proximal end connected to the septum-like structure and a distal end for insertion into the nostrils of a patient. The nasal cannula assembly further comprises a first sealing member adapted to form a fluid connection with the inlet port, the first sealing member having a recess that extends longitudinally along the length thereof such that the recess of the first sealing member conforms to the shape of the lumen of the inlet port and a second sealing member adapted to form a fluid connection with the outlet port.

Preferably, the first length (L1) of the inlet and outlet ports is equal to second length (L2) of the channel.

Preferably, the second inner diameter (Ø2) of the channel is lesser than the first inner diameter (Ø1) of the inlet and outlet ports.

In a preferred embodiment of the present invention, it is disclosed that the pair of nasal prongs, each has an axial partition aligned with the septum-like structure and passing through a wall of the manifold body to intersect the septum-like structure such that gas inhaled by the patient passes from the inlet port only into the nostrils of the patient and gas exhaled by the patient passes at least partially to the outlet port.

Preferably, the pair of nasal prongs comprises a first tube that extends from the inlet chamber for delivering gas into the nostrils of the patient, and a second tube that extends from the outlet chamber for receiving gas exhaled from the nostrils of the patient.

More preferably, the length of the first tube extending from the inlet chamber is shorter than the second tube extending from the outlet chamber.

In another preferred embodiment of the present invention, it is disclosed that the first and second sealing members comprise a sealing element disposed at their proximal end that is fixedly and directly coupled to the inlet port and outlet port, respectively, for sealing thereof.

Preferably, the first and second sealing members, each has an opening that extends therethrough in an axial direction and aligns with the inlet and outlet ports.

Further embodiment of the present invention discloses the nasal cannula assembly further comprises a bulbous-shaped member disposed on top of the channel for positioning adjacent the mouth of the patient to intercept orally exhaled gas and pass at least a sample to the outlet port of the manifold body.

An exemplary embodiment of the present invention describes that the outlet port is connected to a capnograph or any suitable device for measuring the concentration or partial pressure of carbon dioxide exhaled by the patient whereby the device is incorporated with a proper pumping means for drawing the carbon dioxide out during exhalation of the patient, whereas the inlet port is connected to a source of oxygen for delivery oxygen to the nostrils of the patient.

One skilled in the art will readily appreciate that the present invention is well adapted to carry out the objects and obtain the ends and advantages mentioned, as well as those inherent therein. The embodiment described herein is not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 illustrates a top perspective view of a nasal cannula assembly according to the present invention.

FIG. 2 illustrates a side perspective view of a nasal cannula assembly of FIG. 1 according to the present invention.

FIG. 3 illustrates another side perspective view of a nasal cannula assembly of FIG. 1 according to the present invention.

FIG. 4 illustrates a front view of a nasal cannula assembly including the bulbous-shaped member according to the present invention.

FIG. 5 illustrates a cross sectional view of a nasal cannula assembly according to the present invention, taken along the line A-A of FIG. 4.

FIG. 6 illustrates a rear view of a nasal cannula assembly including the bulbous-shaped member according to the present invention.

FIG. 7 illustrates a cross-sectional view of a nasal cannula assembly according to the present invention, taken along the line B-B of FIG. 6.

FIG. 8 illustrates an enlarged view of section C of FIG. 6 depicting further the nasal prongs of a nasal cannula assembly according to the present invention.

FIG. 9 illustrates a front view of the inlet port of a nasal cannula assembly according to the present invention.

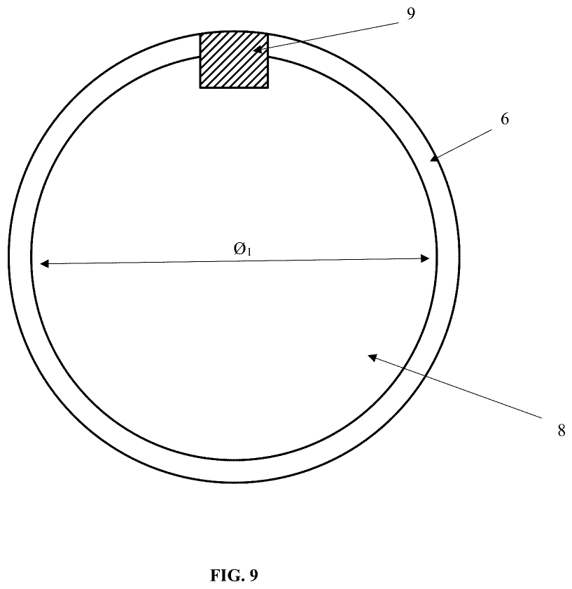

FIG. 10 illustrates a side view of the inlet port of a nasal cannula assembly according to the present invention.

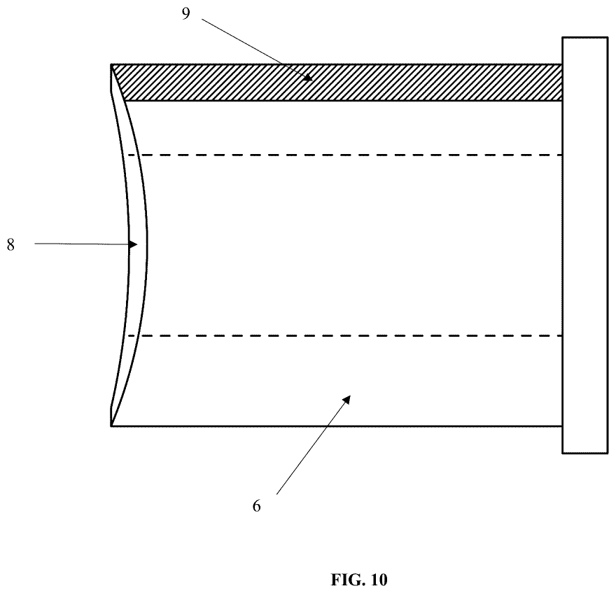

FIG. 11 illustrates a front view of the first sealing member of a nasal cannula assembly according to the present invention.

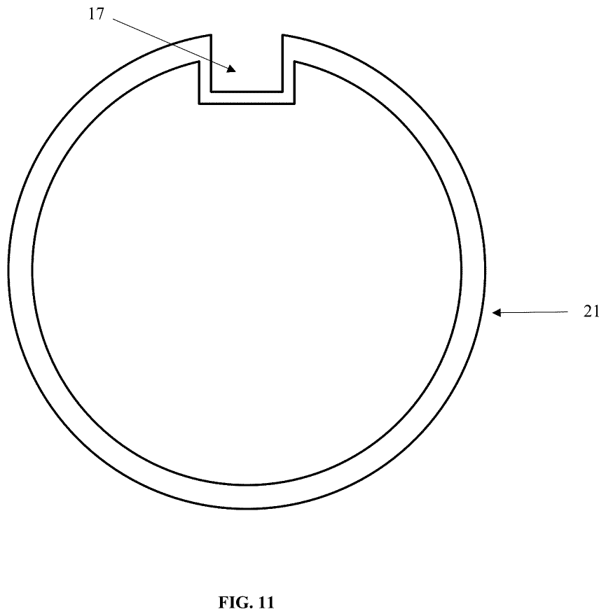

FIG. 12 illustrates a front view of the channel of a nasal cannula assembly according to the present invention.

### DETAILED DESCRIPTION

Hereinafter, the invention shall be described according to the preferred embodiments of the present invention and by referring to the accompanying description and drawings. However, it is to be understood that limiting the description to the preferred embodiments of the invention is merely to facilitate discussion of the present invention and it is envisioned that those skilled in the art may devise various modifications without departing from the scope of the appended claim.

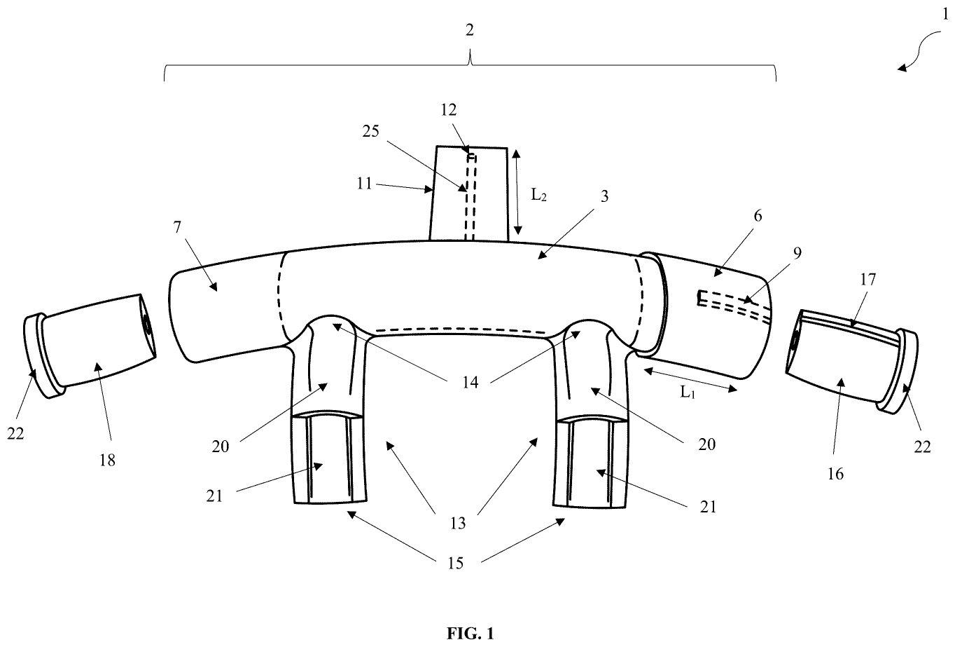

The present invention relates to a nasal cannula assembly suitable for ventilation applications such as continuous positive airway pressure (CPAP) applications, bi-level positive airway pressure (BIPAP) applications and intermittent positive pressure applications. Referring generally to FIGS. 1-3, a device assembled in the form of a nasal cannula (1) that is provided for delivering supply of oxygen from a source into the nostrils of a patient and connected to a carbon dioxide sampling machine is illustrated. The nasal cannula assembly (1) comprises individual components including a manifold body (2), a first sealing member (16) and a second sealing member (18), which can be assembled to form a nasal cannula of the present invention. The aforementioned nasal cannula components may be manufactured from one or more inert materials, such as polyurethane, silicone or the like. In the preferred embodiment, the nasal cannula components of the present invention can typically be assembled or disassembled without the use of any tools, fasteners or adhesives. Alternatively, fasteners or adhesives may be used to assemble the nasal cannula if a more permanent assembly is desired.

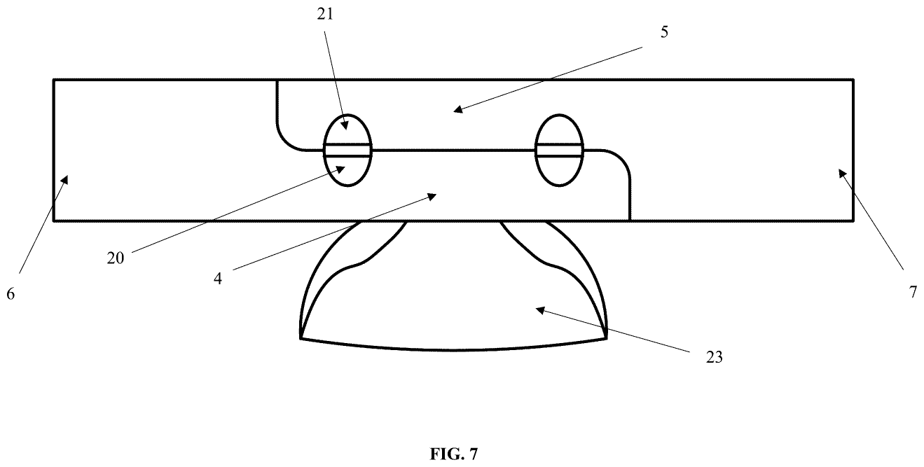

In a preferred embodiment of the present invention, the nasal cannula assembly (1) comprises a main portion of a manifold body (2) having a septum-like structure (3) that is configured to separate an inlet chamber (4) and an outlet chamber (5) that extend generally axially of the manifold body (2) and internally dividing the manifold body (2) into an inlet port (6) and an outlet port (7), as shown in FIGS. 1-3. The separation of the inlet chamber (4) and the outlet chamber (5) is best illustrated in FIG. 7. When using the nasal cannula assembly (1) in hospital environment, the inlet port (6) is typically connected to a source of oxygen such as an ambulatory tank for delivering oxygen to the nostrils of a patient whereas the outlet port (7) can be connected to a capnograph or any suitable machines which is able to measure the concentration or partial pressure of carbon dioxide. For example, such machine may be incorporated with a proper pumping means for drawing carbon dioxide out during breathing.

In a preferred embodiment of the present invention, each of the inlet port (6) and outlet port (7) defines a lumen (8) having a first length (L₁) and a first inner diameter (Ø₁). In the context of the present invention, the lumen (8) refers to the interior space within the tubular structure of the inlet port (6) and outlet port (7). Depending on the compatibility of connection with the oxygen source and carbon dioxide sampling machine, the first length (L₁) of the lumen (8) may be provided in the range of 2 mm to 6 mm. Similarly, the first inner diameter (Ø₁) of the lumen (8) may be provided in the range of 8 mm to 12 mm.

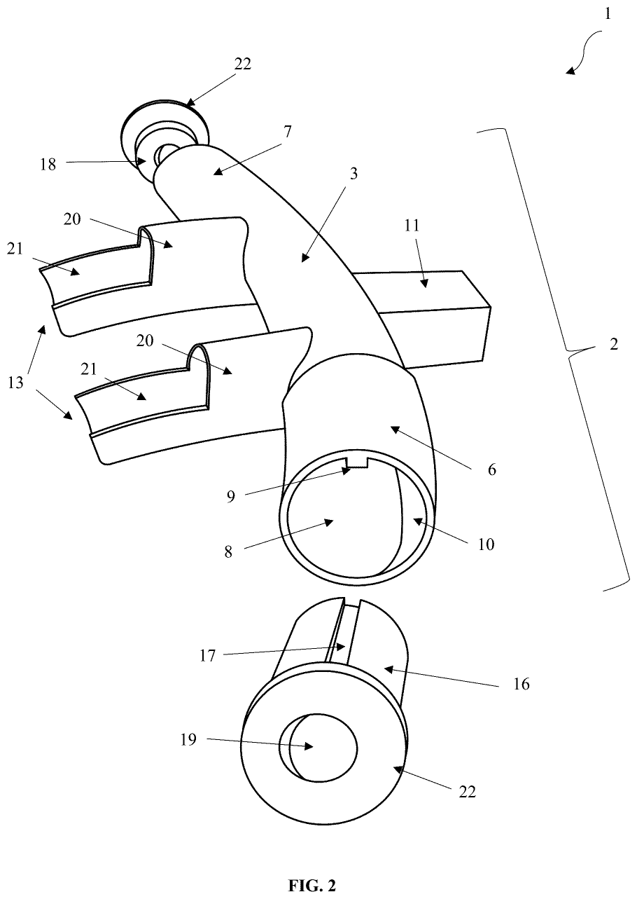

Referring to FIG. 2, the lumen (8) of the inlet port (6) comprises a protrusion (9) being disposed on an inner surface (10) thereof. Preferably, the protrusion (9) is elongated and extends at least partially in the longitudinal direction of the lumen (8). It is to be noted that the presence of the elongated protrusion (9) shall not impede or disrupt the flow of oxygen from the inlet port (6) into the inlet chamber (4). Nevertheless, the elongated protrusion (9) is provided to function as a visual indicator for a medical personnel or professional when attempting to connect the inlet port (6) to the oxygen source. In practice, the visual indication of the elongated protrusion (9) shall prevent misconnection of the inlet port (6) to the wrong route, for example, the carbon dioxide sampling machine or an intravenous catheter. If desired, the elongated protrusion (9) may be pigmented with vibrant colour for easy indication.

Referring to FIGS. 1-3, the manifold body (2) of the nasal cannula assembly (1) comprises a channel (11) that is disposed proximate to the septum-like structure (3) and in fluid communication with the outlet chamber (5). As used herein, the term “proximate” is defined to mean a direction closer to the manifold body (2) of the nasal cannula assembly (1). As seen in FIGS. 1-3, the channel (11) extends in a transverse direction of the septum-like structure (3) and has a second length (L₂). In a preferred embodiment of the present invention, the second length (L₂) of the channel (11) is substantially equal to the first length (L₁) of the inlet port (6) and outlet port (7). It is to be noted that first length (L₁) of the inlet port (6) and outlet port (7) and the second length (L₂) of the channel (11) are relatively shorter than that of conventional nasal cannulas. The reduced length minimizes dead space of the inlet port (6) and outlet port (7) which subsequently reduces or eliminates turbulences adjacent to the opening of the inlet port (6) and outlet port (7), thereby lessens comingling of supplied oxygen and exhaled carbon dioxide to be sampled.

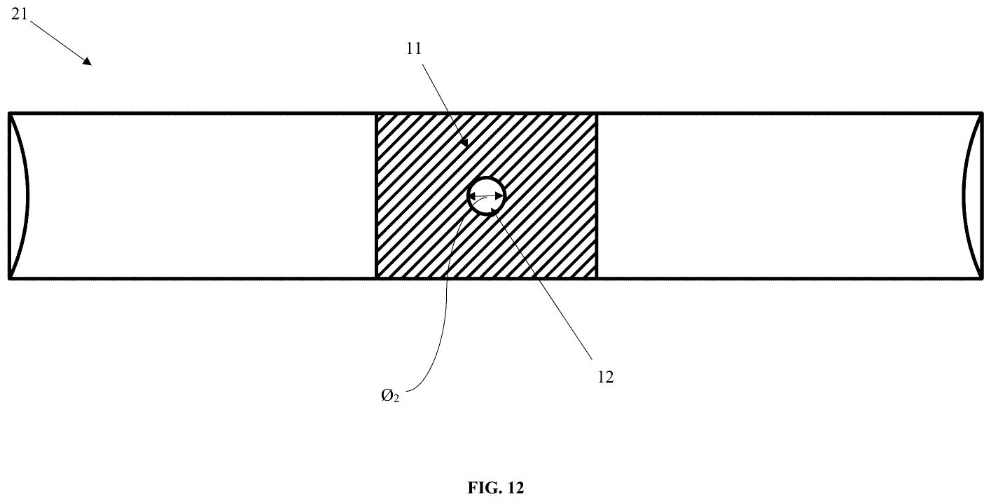

Referring to FIG. 12 illustrating a front view of the manifold body (2), the channel (11) defines an aperture (12) having a second inner diameter (Ø₂) that is substantially lesser than the first inner diameter (Ø₁) of the inlet port (6) and outlet port (7). For example, the second inner diameter (Ø₂) of the aperture (12) may be provided in the range of 1.3 mm to 1.7 mm. Ideally, the substantially smaller aperture (12) of the channel (11) shall also reduce dead space around thereof.

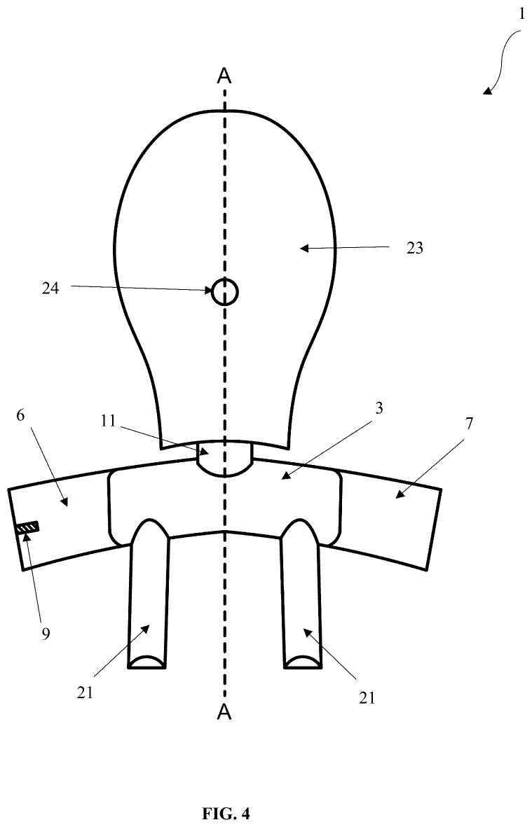

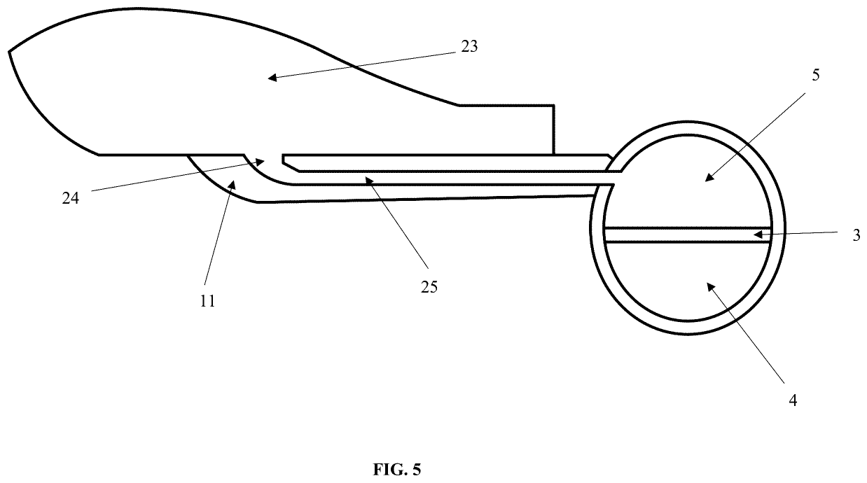

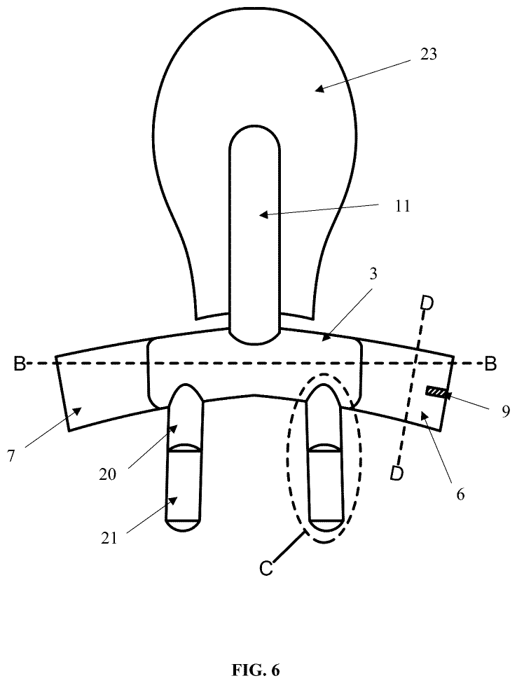

In a preferred embodiment of the present invention, a bulbous-shaped member (23) is disposed on top of the channel (11) having a bore (24) for allowing carbon dioxide gas exhaled by the patient from the mouth to be guided to the outlet chamber (5) via an air passageway (25) located within the channel (11), as illustrated in FIGS. 4-6. The bulbous-shaped member (23) is provided for positioning adjacent the mouth of the patient to intercept orally exhaled carbon dioxide gas and pass at least a sample thereof to the outlet port (7) of the manifold body (2).

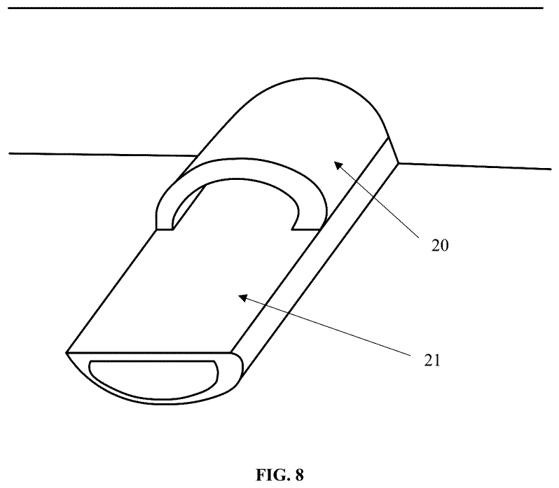

As seen in FIGS. 1-3, the manifold body (2) of the nasal cannula assembly (1) also comprises a pair of nasal prongs (13) having a proximal end (14) connected to the septum-like structure (3) and a distal end (15) for insertion into the nostrils of a patient. As used herein, the term “distal” is defined to mean a direction farther from the manifold body (2) of the nasal cannula assembly (1). Each of the nasal prongs (13) has an axial partition that aligns with the septum-like structure (3) and passes through a wall of the manifold body (2) to intersect the septum-like structure (23), as can be clearly seen in FIG. 7. This allows oxygen gas inhaled by the patient to pass from the inlet port (6) only into the nostrils of the patient and carbon dioxide gas exhaled by the patient to pass at least partially to the outlet port (7). As illustrated in FIG. 1, the pair of nasal prongs (13) comprises a first tube (20) that extends from the inlet chamber (4) for delivering oxygen gas into the nostrils of the patient, and a second tube (21) that extends from the outlet chamber (5) for receiving carbon dioxide gas exhaled from the nostrils of the patient. To adhere to the objective of reducing dead space and therefore minimizing or eliminating breathing turbulence, it is preferred that the length of the first tube (20) extending from the inlet chamber (4) is shorter than the second tube (21) extending from the outlet chamber (5), as seen in FIG. 8.

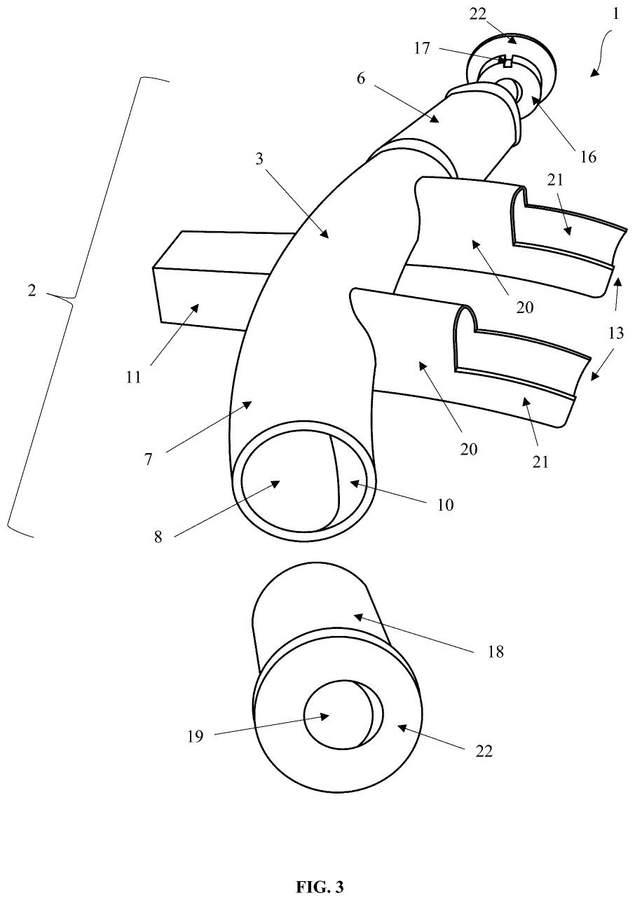

As described above, individual components including a first sealing member (16) and a second sealing member (18) can be assembled to form the nasal cannula (1) of the present invention. In some embodiments, it is further described that the manifold body (2) accommodates and holds adjacent components in a removably or releasably locking or sealing engagement. As such, to form the nasal cannula (1) according to one embodiment of the present invention, the first sealing member (16) is adapted to form a fluid connection with the inlet port (6) of the manifold body (2). For example, the first sealing member (16) can slide onto the inlet port (6) of the manifold body (2) to form a nasal cannula assembly (1) that is held together in a friction fit or mechanical fit. Due to the presence of the elongated protrusion (9) extending on the inner surface of the inlet port (6), the first sealing member (16) is provided with a recess (17) that extends longitudinally along the length thereof such that the recess (17) of the first sealing member (16) conforms to the shape of the lumen (8) of the inlet port (6), as shown in FIG. 2. Further, to form the nasal cannula assembly (1) of the present invention, the second sealing member (18) is also adapted to form a fluid connection with the outlet port (7), as shown in FIG. 3. Similarly, the second sealing member (18) can slide onto the outlet port (7) of the manifold body (2) to form a nasal cannula assembly (1) that is held together in a friction fit or mechanical fit.

To ensure the locking or sealing engagement as abovementioned, the first sealing member (16) and the second sealing member (18) comprise a sealing element (22) being disposed at their proximal end that is fixedly and directly coupled to the inlet port (6) and outlet port (7), respectively. By way of example, the outer circumference of the sealing element (22) may be designed to be substantially larger than the outer circumference of the inlet port (6) and outlet port (7) so that the sealing element (22) abuts against the inlet port (6) and outlet port (7) to fill a gap therebetween, thereby forming the sealing engagement. Ideally, the first sealing member (16) and the second sealing member (18), each has an opening (19) that extends therethrough in an axial direction and aligns with the inlet port (6) and outlet port (7).

The present disclosure includes as contained in the appended claims, as well as that of the foregoing description. Although this invention has been described in its preferred form with a degree of particularly, it is understood that the present disclosure of the preferred form has been made only by way of example and that numerous changes in the details of construction and the combination and arrangements of parts may be resorted to without departing from the scope of the invention.

What is claimed is:

1. A nasal cannula assembly comprising:
a manifold body comprising:
a septum-like structure configured to separate an inlet chamber and an outlet chamber that extend generally axially of the manifold body and internally dividing the manifold body into an inlet port and an outlet port, each defining a lumen having a first length and a first inner diameter;
a channel disposed proximate to the septum-like structure and in fluid connection with the outlet chamber, the channel having a second length and defining an aperture having a second inner diameter; and
a pair of nasal prongs having a proximal end connected to the septum-like structure and a distal end for insertion into the nostrils of a patient.

2. The nasal cannula assembly according to claim 1, wherein the first length of the inlet and outlet ports is equal to the second length of the channel.

3. The nasal cannula assembly according to claim 1, wherein the second inner diameter of the channel is lesser than the first inner diameter of the inlet and outlet ports.

4. The nasal cannula assembly according to claim 1, wherein the lumen of the inlet port comprises a protrusion disposed on an inner surface thereof that extends at least partially in the longitudinal direction of the lumen.

5. The nasal cannula assembly according to claim 1 further comprising a first sealing member adapted to form a fluid connection with the inlet port, wherein the first sealing member has a recess that extends longitudinally along the length thereof such that the recess of the first sealing member conforms to the shape of the lumen of the inlet port.

6. The nasal cannula assembly according to claim 1 further comprising a second sealing member adapted to form a fluid connection with the outlet port.

7. The nasal cannula assembly according to claim 1, wherein the first and second sealing members comprise a sealing element disposed at their proximal end that is fixedly and directly coupled to the inlet port and outlet port, respectively, for sealing thereof.

8. The nasal cannula according to claim 1, wherein the first and second sealing members, each has an opening that extends therethrough in an axial direction and aligns with the inlet and outlet ports.

9. The nasal cannula assembly according to claim 1, wherein the pair of nasal prongs, each has an axial partition aligned with the septum-like structure and passing through a wall of the manifold body to intersect the septum-like structure such that gas inhaled by the patient passes from the inlet port only into the nostrils of the patient and gas exhaled by the patient passes at least partially to the outlet port.

10. The nasal cannula assembly according to claim 1, wherein the pair of nasal prongs comprises a first tube that extends from the inlet chamber of the septum-like structure for delivering gas into the nostrils of the patient, and a second tube that extends from the outlet chamber of the septum-like structure for receiving gas exhaled from the nostrils of the patient.

11. The nasal cannula assembly according to claim 1, wherein the length of the first tube extending from the inlet chamber of the septum-like structure is shorter than the second tube extending from the outlet chamber of the septum-like structure.

12. The nasal cannula assembly according to claim 1 further comprising a bulbous-shaped member disposed on top of the channel for positioning adjacent the mouth of the patient to intercept orally exhaled gas and pass at least a sample to the outlet port of the manifold body.

13. The nasal cannula assembly according to claim 1, wherein the outlet port is adapted to be connected to a capnograph or any suitable device for measuring the concentration or partial pressure of carbon dioxide exhaled by the patient.

14. The nasal cannula assembly according to claim 1, wherein the inlet port is connected to a source of oxygen for delivery oxygen to the nostrils of the patient.
